(19) United States
(12) Patent Application Publication
(10) Pub. No.: US 2019/0365694 A1
(43) Pub. Date: Dec. 5, 2019

(54) COMPOSITION OF MATTER AND USE THEREOF FOR PREVENTING AND TREATING MOTION SICKNESS

(71) Applicant: GT BIOPHARMA, INC., Westlake Village, CA (US)

(72) Inventor: Kathleen E. CLARENCE-SMITH, Washington, DC (US)

(73) Assignee: GT BIOPHARMA, INC., Westlake Village, CA (US)

(21) Appl. No.: 16/474,683

(22) PCT Filed: Dec. 29, 2017
(86) PCT No.: PCT/US17/68944

§ 371 Date: Jun. 28, 2019

Related U.S. Application Data
(60) Provisional application No. 62/595,667, filed on Dec. 7, 2017.
(60) Provisional application No. 62/440,575, filed on Dec. 30, 2016.

(51) Int. Cl. A61K 31/216; A61K 9/70; A61P 1/08

(52) U.S. Cl. CPC A61K 31/216; A61P 1/08; A61K 9/7023

(57) ABSTRACT

Motion sickness in a mammalian subject, in particular, a human, may be prevented or treated by administering to a mammalian, a transdermal device system releasing oxybutynin or one of its enantiomers, such as (R)-oxybutynin.

### RELATED APPLICATIONS

This application claims the benefit of U.S. Provisional Patent Application Ser. No. 62/440,575, filed Dec. 30, 2016, and U.S. Provisional Patent Application Ser. No. 62/595,667, filed Dec. 7, 2017, the disclosures of which are incorporated herein in their entirety by reference.

### FIELD OF THE INVENTION

This invention pertains to the field of the treatment of motion sickness and a composition useful for safely preventing and/or treating motion sickness.

### BACKGROUND OF THE INVENTION

Motion sickness is a well-known syndrome that typically involves nausea and vomiting upon exposure to certain types of motion, in particular upon exposure to swinging, turning, rocking, or up-and-down movements (Encyclopedia Britannica). It is considered to be of central origin and caused by a conflict between the vestibular, visual, and other proprioceptive systems (Besthaven et al., 2016). In addition to nausea and vomiting, other symptoms may include pallor, cold sweats, hypersalivation, hyperventilation and headaches (Spinks et al, 2004). Although nausea is the hallmark symptom, it is often preceded by stomach awareness, malaise, drowsiness, and irritability (Brainard and Gresham, 2014).

Motion sickness occurs in both humans and animals (Schmal, 2013) during travel by sea, automobile, airplane, helicopter, and in space (Estrada et al, 2007). Furthermore, some other special situations, such as simulators, the cinema, and video games, have been described as causing “pseudo-motion sickness” (Schmal, 2013). Children between 2 and 12 years old are most susceptible to motion sickness, and women are more frequently affected than men (Schmal, 2013). Estimates of the prevalence of motion sickness varies from 9% to 38% of the population, depending on the studies (Koslucher et al, 2015).

Seasickness, a form of motion sickness, is a widespread problem among sailors, and has a major impact on their performance at sea (Shupak et al, 1989). Severe motion sickness is a huge obstacle for people conducting precise aviation, marine, or emergency service tasks (Zhang et al., 2016). Motion sickness remains bothersome in conventional transport and is an emerging hazard in visual information technologies. Treatment remains unsatisfactory.

First-generation antihistamines alone, for example diphenhydramine, or in a fixed-dose combination with a xanthine derivative such as 8-chlorotheophylline (dimenhydrinate, known as Dramamine®), are effective against motion sickness, but are sedating. Non-sedating antihistamines, ondansetron, and ginger root are not effective in the prevention and treatment of motion sickness (Brainard, 2014).

Scopolamine remains the most effective single drug for the prophylaxis and treatment of motion sickness (Parrot, 1989). However, oral or injected scopolamine displays a comparatively short duration of action (5-6 hours), and leads to deleterious side effects on autonomic and central nervous system cholinergic functions. These adverse effects in young and very young people suffering from motion sickness could be particularly worrying (Lin et al., 2011).

The transdermal scopolamine system was designed to reduce these problems. Transdermal scopolamine is a first-line medication for prevention of motion sickness and should be administered several hours before the anticipated motion exposure (Gil et al., 2012; Brainard, 2014).

While transdermal scopolamine delivers scopolamine over a prolonged time period (72 hours), deleterious side effects continue to be produced. Central nervous system effects are of particular concern and include sedation (Spinks, 2004), reduced memory for new information, impaired attention, and lowered feelings of alertness (Parrot, 1989). Mental confusion or delirium can occur after application of the scopolamine patch (Seo et al., 2009). Elderly people as well as people with undetected incipient dementia or mild cognitive impairment (MCI) may be particularly prone to develop mental confusion after applying the scopolamine patch (Seo et al., 2009). The cognitive adverse effects of transdermal scopolamine are also of particular concern during space travel (Paule, 2004). Space motion sickness (SMS) is a problem during the first 72 hours of space flight and during transitions from different gravity environments. There currently are no effective drug countermeasures for SMS that also accommodate the retention of optimal cognitive function. This creates a dilemma for astronauts because cognitive skills are particularly important during gravity transitions (e.g., take-off and landing). Clinical trial data suggest that, at clinically useful doses, the rank order of the drugs with the best cognitive profiles is meclizine>scopolamine>promethazine>lorazepam (Paule, 2004).

Central anticholinergic syndrome has also been reported following the use of scopolamine patch (Holland, 1992). Transdermal scopolamine can affect postural control and the perception of verticality, thereby increasing the risk of falls, especially in the elderly, people with Parkinson's disease, and people with other neurodegenerative disorders. Falls are also problematic in sailors and in parabolic flight (Besthaven et al., 2016).

Other side effects reported with scopolamine include delirium due to scopolamine patch in a 4-year-old boy, described by Lin Y G et al. (2001); and withdrawal symptoms after discontinuation of transdermal scopolamine therapy, needing a treatment with meclizine, as described by Patel and Ezzo (2009).

Scopolamine is a muscarinic receptor antagonist that exhibits similar binding affinities to all of the five known muscarinic receptor sub-types (Golding and Stott, 1997). The drug has been demonstrated to have high selectivity for the muscarinic receptor [Hulme E C, Birdsall N J, Burgen A S, Mehta P (1978) The binding of antagonists to brain muscarinic receptor. Mol Pharmacol 14: 737-750.], although it has been reported to block nicotinic receptors when administered in high doses [Schmeller T, Sporer F, Sauerwein M, Wink M (1995) Binding of tropane alkaloids to nicotinic and muscarinic acetylcholine receptors. Pharmazie 50: 493-495.]. Scopolamine acts both centrally and peripherally.

Scopolamine is thought to exert its anti-motion sickness effects by acting on the vestibular nucleus and potentially on the vestibulo-spinal pathway, which may affect balance and motor tasks requiring both attentional process and motor balance (Besthaven et al., 2016).

A quaternary derivative of scopolamine, methyl scopolamine bromide (“methscopolamine”), that has been shown to virtually not penetrate the Blood Brain Barrier (FDA Label), has also been reported to be effective for the prevention and treatment of motion sickness, but is less potent than scopolamine (Spinks, 2011), suggesting that the efficacy of scopolamine for the treatment of motion sickness could involve, at least in part, muscarinic receptors that are located outside the BBB. Methscopolamine, however, is less potent than scopolamine, is poorly absorbed from the gut, and is not absorbed from the skin (FDA Label), making the use of transdermal patches practically impossible.

Another scopolamine quaternary salt of scopolamine, butylscopolamine bromide, seems to have been used off-label for alleviating the motion sickness syndrome, but it has the same drawbacks as those of methscopolamine.

Thus, the problem of the central adverse effects of the available anti-motion-sickness products remains unsolved and there is a need for an efficacious anti-motion-sickness medication that, while preventing and combating motion sickness, does not involve the central adverse effects that can render the treatment with transdermal scopolamine not only difficult to tolerate, but even dangerous.

### SUMMARY OF THE INVENTION

The present invention relates to a non-selective peripheral anticholinergic agent as a good anti-motion sickness medication, preferably not acting via oral route, in order to assure the safe prevention and/or treatment of the disorder, as a subject suffering of motion sickness has noteworthy difficulty in taking oral drugs.

The present inventor found that transdermal oxybutynin efficaciously prevents and treats motion sickness.

The present invention provides, in a preferred embodiment, a TDDS comprising a racemic (R/S)-oxybutynin or non-racemic mixture of (R)-oxybutynin and (S)-oxybutynin, formulated with a pharmaceutical carrier or vehicle, said TDDS releasing from 0.78 mg/24 h to 31.2 mg/24 h of said racemic (R/S)-oxybutynin or from 0.78 mg/24 h to 15.6 mg/24 h of (R)-oxybutynin in said non-racemic mixture of (R)-oxybutynin and (S)-oxybutynin.

The present invention also provides, a TDDS comprising a non-racemic mixture of (R)-oxybutynin and (S)-oxybutynin, formulated with a pharmaceutical carrier or vehicle, said TDDS releasing from 0.78 mg/24 h to 15.6 mg/24 h of said racemic (R/S)-oxybutynin or releasing from 0.78 mg/24 h to 8 mg/24 h of (R)-oxybutynin in said non-racemic mixture of (R)-oxybutynin and (S)-oxybutynin.

The present invention is also based on the fact that, contrary to, for example, oral oxybutynin, in a TDDS substantially acts as a non-selective, peripheral anticholinergic agent.

The present inventor found that (R)-4-diethylaminobut-2-ynyl 2-cyclohexyl-2-hydroxy-2-phenylethanoate, herein referred to as “(R)-oxybutynin”, when administered transdermally, particularly efficaciously prevents and treats motion sickness.

In the context of the present invention, the expression “TDDS-(R)-oxybutynin” refers to a transdermal drug delivery system, such as a patch, comprising (R)-oxybutynin, in admixture with a pharmaceutical carrier or vehicle for the transdermal delivery.

In particular a TDDS-(R)-oxybutynin may be a safer alternative to the scopolamine patch for the treatment of motion sickness, especially in pediatric patients and in patients who do not tolerate the scopolamine patch, in particular in elderly people patients with undetected MCI or suffering from Alzheimer's disease.

Thus, the present invention provides a method for preventing and/or treating motion sickness in a mammalian subject in need thereof, which comprises administering to said mammalian a transdermal drug delivery system (TDDS) releasing (R)-oxybutynin and (S)-oxybutynin, as an active principle.

Preferably, the present invention provides a method for preventing and/or treating motion sickness in a human subject, which comprises administering to said human subject a transdermal drug delivery system (TDDS) releasing (R)-oxybutynin and (S)-oxybutynin, as an active principle.

In addition, the present invention provides a method for preventing and/or treating motion sickness in a mammalian in need thereof, which comprises administering to said mammalian a TDDS releasing (R)-oxybutynin, as an active principle.

Preferably, the present invention provides a method for preventing and/or treating motion sickness in a human subject, which comprises administering to said human subject a TDDS releasing (R)-oxybutynin, as an active principle.

The present invention further provides, a TDDS comprising (R)-oxybutynin, formulated with a pharmaceutical carrier or vehicle, said TDDS releasing from 0.78 mg/24 h to 15.6 mg/24 h of said (R)-oxybutynin.

Preferably, the invention provides, a TDDS comprising (R)-oxybutynin, formulated with a pharmaceutical carrier or vehicle, said TDDS releasing from 0.78 mg/24 h to 4 mg/24 h of said (R)-oxybutynin.

In a first embodiment, the present invention relates to a method for preventing and/or treating motion sickness in a mammalian subject in need thereof, which involves administering to said mammalian, a transdermal drug delivery system (TDDS) comprising as an active ingredient, (R)-oxybutynin and (S)-oxybutynin. The (R)-oxybutynin and (S)-oxybutynin may be present as a racemic mixture or a non-racemic mixture.

Preferably, the invention relates to a method for preventing and/or treating motion sickness in a human subject in need thereof, which involves administering to said human subject, a transdermal drug delivery system (TDDS) comprising as an active ingredient, (R)-oxybutynin and (S)-oxybutynin. The (R)-oxybutynin and (S)-oxybutynin may be present as a racemic mixture or a non-racemic mixture.

The TDDS releases said non-racemic mixture in an amount of from 0.78 mg/24 h to 15.6 mg/24 h of (R)-oxybutynin in said non-racemic mixture, or in an amount of from 0.78 mg/24 h to 8 mg/24 h of (R)-oxybutynin in said non-racemic mixture. Preferably, in an amount of 3.9 mg/24 h of (R)-oxybutynin in said non-racemic mixture.

The TDDS releases said racemic mixture in an amount of from 0.78 mg/24 h to 31.2 mg/24 h, or in an amount of from 0.78 mg/24 h to 15.6 mg/24 h. Preferably, in an amount of 8 mg/24 h.

In a second embodiment, the present invention relates to a method for preventing and/or treating motion sickness in a mammalian subject in need thereof, which involves administering to said mammalian subject, a transdermal drug delivery system (TDDS) comprising as an active ingredient, (R)-oxybutynin. The TDDS may be substantially free of (S)-oxybutynin, or comprise traces of (S)-oxybutynin.

Preferably, the present invention relates to a method for preventing and/or treating motion sickness in a human subject in need thereof, which involves administering to said human subject, a transdermal drug delivery system (TDDS) comprising as an active ingredient, (R)-oxybutynin. The TDDS may be substantially free of (S)-oxybutynin, or comprise traces of (S)-oxybutynin.

The TDDS releases said (R)-oxybutynin as an active ingredient in an amount of from 0.78 mg/24 h to 15.6 mg/24 h, or in an amount of from 0.78 mg/24 h to 4 mg/24 h. Preferably, in an amount of 3.9 mg/24 h.

The TDDS useful in the methods of the invention may further include a pharmaceutical carrier or vehicle.

In a third embodiment, the invention relates to a transdermal drug delivery system containing (R)-oxybutynin, said transdermal drug delivery system releasing from 0.78 mg/24 h to 15.6 mg/24 h of said (R)-oxybutynin. The TDDS may release from 0.78 mg/24 h to 4 mg/24 h of said (R)-oxybutynin.

Preferably, the TDDS may be free of (S)-oxybutynin or contain traces of (S)-oxybutynin.

In a fourth embodiment, the invention relates to a transdermal drug delivery system containing (R)-oxybutynin and (S)-oxybutynin

In particular, the TDDS contains (R)-oxybutynin and (S)-oxybutynin as a racemic mixture, or contains a non-racemic mixture of (R)-oxybutynin and (S)-oxybutynin.

The TDDS containing a racemic mixture releasing from 0.78 mg/24 h to 31.2 mg/24 h of said racemic (R)-oxybutynin and (S)-oxybutynin mixture, preferably, from 0.78 mg/24 h to 8 mg/24 h of said racemic (R)-oxybutynin and (S)-oxybutynin mixture. The TDDS containing the racemic mixture may be a patch, a patch pump, an infusion pump, or a micropump.

The TDDS containing a non-racemic mixture releasing from 0.78 mg/24 h to 15.6 mg/24 h of (R)-oxybutynin in said non-racemic (R)-oxybutynin and (S)-oxybutynin mixture, preferably, from 0.78 mg/24 h to 8 mg/24 h of (R)-oxybutynin in said non-racemic (R)-oxybutynin and (S)-oxybutynin mixture. The TDDS containing the non-racemic mixture may be a patch, a patch pump, an infusion pump, or a micropump.

The TDDS containing a non-racemic mixture of (R)-oxybutynin and (S)-oxybutynin in which (R)-oxybutynin is present from more than 50% to 95% by weight based on the total weight of the mixture.

Alternatively, the TDDS contains a non-racemic mixture of (R)-oxybutynin and (S)-oxybutynin in which (S)-oxybutynin is present from 5% to less than 50% by weight based on the total weight of the mixture, from 40% or less by weight based on the total weight of the mixture, from 30% or less, from 20% or less, or from 10% or less by weight based on the total weight of the mixture.

### DEFINITIONS

“Peripheral”: applies to anticholinergics that are largely unable (have a limited ability) to enter the central nervous system following systemic administration and thus do not affect brain function to a clinically appreciable degree. These drugs can include both quaternary and tertiary ammonium anticholinergic agents, especially those having low lipid solubility.
“Transdermal delivery” of drug can be targeted to skin tissues just under the skin, regional tissues or organs under the skin, systemic circulation, and/or the central nervous system.
“Transdermal drug delivery system” provides transdermal delivery using transdermal drug formulations and transdermal patches incorporating such transdermal drug formulations. For example, the transdermal drug delivery system may include a composition in form of a patch, a cream, a gel, a lotion or a paste comprising (R)-oxybutynin. Alternatively, the transdermal drug delivery system may include a composition in form of a patch, a cream, a gel, a lotion or a paste comprising a racemic (R/S)-oxybutynin or non-racemic mixture of (R)-oxybutynin and (S)-oxybutynin. For example, transdermal formulations may include, but are not limited, to those as described in U.S. Pat. No. 6,562,368, a transdermal gel formulation as described in U.S. Pat. Nos. 7,029,694; 7,179,483; 8,241,662 and US 2009/0018190, a transdermal or transmucosal pharmaceutical formulation, that can be utilized for topical or transdermal application, such that solutions, creams, lotions, sprays, ointment, gels, aerosols and patch drug deliveries as described in WO 2005/039531, US2007/022379, US 2010/0216880, US 2014/0037713 and U.S. Pat. No. 8,652,491, a transdermal absorption preparation as described in WO2013/061969 and US 2014/0271796, the disclosures of which are herein incorporated by reference in their entirety. The transdermal patches may also include, but are not limited to, a patch pump having an in-dwelling rigid catheter with flexible features and/or a flexible catheter attachment as described in U.S. Pat. No. 9,782,536, a selectively activatable patch pump as described in U.S. Pat. No. 9,724,462, a patch pump attached to a wireless communication system as described in U.S. Pat. No. 9,623,173, a conformable patch pump as described in U.S. Pat. No. 9,616,171, an infusion pump as described in U.S. Pat. No. 8,915,879, a portable infusion drug delivery as described in U.S. Pat. No. 8,480,649, a micropump as described in U.S. Pat. No. 8,282,366, and a patch pump as described in U.S. Pat. No. 7,828,771; the disclosures of which are herein incorporated by reference in their entirety. Other transdermal patches may include, but are not limited to, a patch in which oxybutynin is incorporated in an adhesive agent layer composition comprises the acrylic-based polymer as the adhesive base agent, and the acrylic-based polymer is a copolymer of polymethyl methacrylate with a polyacrylateas described in U.S. Pat. No. 8,802,134, a patch consisting of a support layer and of an adhesive agent layer arranged on the at least one surface of the support layer as described in U.S. Pat. No. 8,877,235, a patch using a monoglyceride or a mixture of monoglycerides of fatty acids as skin permeation-enhancer as described in U.S. Pat. No. 5,441,740 and U.S. Pat. No. 5,500,222, a patch for using a monoglyceride or a mixture of monoglycerides plus a lactate ester as skin permeation-enhancer as described in U.S. Pat. Nos. 5,686,097; 5,747,065; 5,750,137 and 5,900,250, a patch with a non-rate controlling tie layer on the skin-proximal surface of the reservoir, not affecting the drug release as described in U.S. Pat. Nos. 5,614,211 and 5,635,203, a patch using triacetin as permeation enhancer as described in U.S. Pat. Nos. 5,212,199, 5,227,169, 5,601,839 and 5,834,010, a patch with a matrix mass in the form of a layer which is self-adhesive, and in which the matrix mass consists of ammonium-group-containing (meth)acrylate copolymers as described in U.S. Pat. No. 6,555,129, a transdermal patch as described in U.S. Pat. Nos. 6,743,441; 7,081,249; 7,081,250; 7,081,251; 7,081,252 and 7,087,241; the disclosures of which are herein incorporated by reference in their entirety.

“Medicament” refers to a substance used in therapy, or a substance that is known or regarded as effective in bringing about recovery or restoration of health of the normal functioning of the body. The medicament may include (R)-oxybutynin and traces of (S)-oxybutynin, or the medicament may include (R)-oxybutynin that is free from the (S)-enantiomer. The medicament may also include a racemic (R/S)-oxybutynin or non-racemic mixture of (R)-oxybutynin and (S)-oxybutynin. The medicament may be, but is not limited to, a transdermal drug formulation or transdermal patch incorporating such transdermal drug formulations for use in a TDDS as described herein.

### DETAILED DESCRIPTION

As outlined above, motion sickness is generally considered to be a Central syndrome; therefore, peripheral anticholinergics should not work. Hence, the use of scopolamine (a centrally- and peripherally-acting anticholinergic), with all the attending aforementioned centrally-mediated side effects (such as, sedation; impaired cognition; in pre-Alzheimer patients, the precipitation of transient dementia; and falls).

Instead, the present invention is based on a new interpretation of the genesis of motion sickness, in particular, that the cause of this disorder is not of a striking central origin, but is peripherally mediated.

The cause of motion sickness is incompletely understood, but the vestibular systems seems to be involved (Besthaven, 2016). Scopolamine, known to be the most effective therapy to control motion sickness, is thought to exert its anti-motion sickness effects by blocking muscarinic receptors on vestibular nuclei in the brain stem (Besthaven et al, 2016). Scopolamine remains the most effective single drug for the prophylaxis and treatment of motion sickness (Parrot, 1989). However, oral or injected scopolamine displays a comparatively short duration of action (5-6 hours), and by its effects on the brain, leads to deleterious side effects. The transdermal scopolamine system was designed to reduce these problems by blunting Cₘₐₓ and overcoming the short duration of action.

Today, transdermal scopolamine is a first-line medication for prevention of motion sickness (Gil et al, 2012; Brainard, 2014)). First-generation antihistamines, although sedating, are also effective. Non-sedating antihistamines, ondansetron, and ginger root are not effective in the prevention and treatment of motion sickness (Brainard, 2014). While transdermal scopolamine delivers scopolamine over a prolonged time period (72 hours), deleterious side effects continue to be produced. Central nervous system effects are of particular concern and comprise sedation (Spinks, 2004), reduced memory, impaired attention, and lowered feelings of alertness (Parrot, 1989). Mental confusion or delirium can occur after application of scopolamine patch (Seo et al, 2009). Elderly people as well as people with undetected incipient dementia or mild cognitive impairment (MCI) may be particularly prone to develop mental confusion after applying the scopolamine patch (Seo et al, 2009). Central anticholinergic syndrome has also been reported following the use of scopolamine patch (Holland 1992).

Scopolamine is a muscarinic receptor antagonist that exhibits similar binding affinities to all of the five known muscarinic receptor sub-types (Golding and Stott, 1997). The drug has been demonstrated to have high selectivity for the muscarinic receptor (Hulme et al, 1978). Scopolamine crosses the Blood Brain Barrier (BBB), and acts both in the brain, inside the BBB, and outside the BBB. Its action on muscarinic receptors in the brain (inside the BBB), explains the deleterious effects on memory, attention, and alertness. Its effects on muscarinic receptors in the vestibular nuclei are thought to explain the anti-motion sickness efficacy (Besthaven et al, 2016).

Many believe that the vestibular nuclei on which scopolamine acts to prevent motion sickness are within the BBB. Thus, most believe that to prevent motion sickness, an anti-muscarinic drug must cross the BBB. Accordingly, the deleterious side effects on memory, attention, and alertness are an unavoidable consequence of the anti-motion sickness efficacy.

However, a quaternary derivative of scopolamine, methscopolamine, that has been shown to virtually not penetrate the Blood Brain Barrier [Pamine® (methscopolamine) Tablets Prescribing Information, September 2012], has also been reported to be effective for the prevention and treatment of motion sickness, but is less potent than scopolamine (Spinks and Wasiak, 2011), suggesting that the efficacy of scopolamine for the treatment of motion sickness could involve—at least in part—muscarinic receptors that are located outside the BBB. Methscopolamine, however, is poorly absorbed from the gut, and it is not absorbed from the skin [[Pamine® (methscopolamine) Tablets Prescribing Information, September 2012], making the use of transdermal patches impossible.

Oxybutynin, is a muscarinic receptor antagonist that has been shown to have few central nervous system adverse effects when administered transdermally, although it penetrates the Blood Brain Barrier, as evidenced by ¹⁴C studies. Notably, the incidence of sedation with oxybutynin TDDS is low (Kennelly, 2010). Furthermore, oxybutynin TDDS does not affect cognition (Kay et al, 2009). Oxybutynin has also been shown to prevent nausea and vomiting.

Accordingly, without being bound by theory, oxybutynin TDDS is believed to be a safer alternative to the scopolamine patch for the treatment of motion sickness. Oxybutynin TDDS is approved by the FDA for the treatment of Overactive Bladder disease. The daily recommended dose in this indication is 3.9 mg/day. However, because the current patch size for oxybutynin TDDS is 39 cm², the administration of higher doses of oxybutynin (that might be needed) is difficult and impractical.

The present invention is a method for preventing and/or treating motion sickness in a mammalian subject in need thereof, which comprises treating said mammalian with a transdermal delivery device system for oxybutynin or one of its enantiomers (TDDS-oxybutynin). Preferably, the invention is a method preventing and/or treating motion sickness in a human subject in need thereof, which comprises treating said human subject with a transdermal delivery device system for oxybutynin or one of its enantiomers (TDDS-oxybutynin).

Oxybutynin is the International Non-proprietary Name (INN) of the racemic 4-diethylaminobut-2-ynyl 2-cyclohexyl-2-hydroxy-2-phenylethanoate of formula

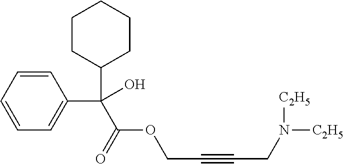 [SMILES: CCN(CC)CC#CCOC(=O)C(O)(c1ccccc1)C1CCCCC1]

described, by its synonym 4-diethylamino-2-butynil phenylcyclohexylglycolate, as hydrochloride, in U.S. Pat. No. 3,176,019 and GB 940,540, the contents of which are incorporated herein in their entirety for reference. Oxybutynin is a chiral compound with its chiral centre in the alpha-carbon atom of the glycolic acid [═C*(OH)—CO—O—] moiety.

The (R) and the (S) enantiomers of oxybutynin were prepared and evaluated for their antimuscarinic, Ca⁺⁺-channel antagonistic and spasmolytic effects in guinea pig detrusor strips and ileal longitudinal muscle by Kachur et al. (1988).

The dextrorotatory form of oxybutynin, (S)-oxybutynin, has been shown to provide a superior therapy in treating urinary incontinence, as disclosed in U.S. Pat. Nos. 5,532,278 and 5,677,346, the contents of which are incorporated therein in their entirety for reference.

In addition, according to Noronha-Blob et al. (1991) oxybutynin and its enantiomers exert moderately potent and stereoselective antagonistic effects at all the tested muscarinic receptor subtypes. Furthermore, the activity of racemic oxybutynin on gastrointestinal motility is attributable to the (R) enantiomer (see also WO 98/01125, the contents of which are incorporated herein in their entirety for reference).

Thus, in its use targeting peripheral muscarinic receptors, each of racemic oxybutynin, (S)-oxybutynin and (R)-oxybutynin, is active on said receptors, thus being a useful active ingredient of a transdermal device system for treating motion sickness.

In the context of the present invention, the expression “TDDS-oxybutynin” may refer to a transdermal drug delivery system, such as a patch, comprising an active ingredient selected from the group consisting of racemic oxybutynin also referred to as “(R/S)-oxybutynin”, (R)-oxybutynin, (S)-oxybutynin, and a non-racemic mixture of (R)-oxybutynin and (S)-oxybutynin, in admixture with a pharmaceutical carrier or vehicle. In addition, the expression “TDS-oxybutynin” may also refer to a transdermal drug delivery system, such as a patch, comprising (R)-oxybutynin, in admixture with a pharmaceutical carrier or vehicle.

(R)-oxybutynin, its preparation and its use for treating urinary incontinence are disclosed in U.S. Pat. No. 6,123,961, the contents of which are incorporated herein in their entirety for reference.

The stereoselectivity of the enantiomers of oxybutynin was studied in guinea pig bladder and ileum in vitro and in vivo (Kachur et al, 1988). In vitro, the affinity of R-oxybutynin for muscarinic receptors was significantly higher than that of S-oxybutynin. A similar significant difference in potency was observed in vivo between the R and the S-isomer.

In addition, according to Noronha-Blob et al. (1991) (R)-oxybutynin exerts moderately potent and stereoselective antagonistic effects at all the tested muscarinic receptor subtypes. Furthermore, the activity of racemic oxybutynin on gastrointestinal motility is attributable to the (R) enantiomer (see also WO 98/01125, the contents of which are incorporated herein in their entirety for reference).

Thus, in its use targeting peripheral muscarinic receptors, (R)-oxybutynin is active on said receptors, thus being a useful active ingredient to be formulated in a transdermal drug delivery system for treating motion sickness.

Motion sickness may include, but is not limited to mal de debarquement (MdDS) and mal de terre. MdDS is is typified by a prolonged rocking sensation that begins immediately following a lengthy exposure to motion, notably, the provoking motion is usually a sea voyage (Hain TC. And Cherchi M., Hand. Clin Neurol., 137: 391-395 (2016). Unlike dizziness caused by vestibular disorders or motion sickness, the symptoms of MdDS usually improve with re-exposure to motion, and the long duration of symptoms distinguishes MdDS from land-sickness (Hain T C. And Cherchi M., Hand. Clin Neurol., 137: 391-395 (2016).

The fact that TDS-oxybutynin provides the prevention and/or the treatment of motion sickness is demonstrated by a pilot study, as described below, using the commercially available oxybutynin patch releasing 3.9 mg/24 h oxybutynin.

According to one embodiment, the invention provides a TDDS comprising a racemic (R/S)-oxybutynin or non-racemic mixture of (R)-oxybutynin and (S)-oxybutynin, said TDDS releasing from 0.78 mg/24 h to 31.2 mg/24 h of said racemic (R/S)-oxybutynin, or said TDDS releasing from 0.78 mg/24 h to 15.6 mg/24 h of (R)-oxybutynin in said non-racemic mixture of (R)-oxybutynin and (S)-oxybutynin. The TDDS may also be formulated with a pharmaceutical carrier or vehicle.

According to another embodiment, the invention provides a TDDS comprising (R)-oxybutynin, said TDDS releasing from 0.78 mg/24 h to 15.6 mg/24 h of said (R)-oxybutynin, preferably from 0.78 mg/24 h to 4 mg/24 h of said (R)-oxybutynin. The TDDS may also be formulated with a pharmaceutical carrier or vehicle. The TDDS may include traces of (S)-oxybutynin.

The expression “traces of (S)-oxybutynin” refers to an amount lower than 5%, advantageously, lower than 2%, advantageously from 1% to 0.1%, normally lower than 0.1%. Alternatively, the (R)-oxybutynin is free from the (S)-enantiomer.

Accordingly, the TDDS in some embodiments may be free of (S)-oxybutynin, or contain traces of (S)-oxybutynin.

According to another embodiment, the invention provides, a TDDS comprising a racemic (R/S)-oxybutynin or non-racemic mixture of (R)-oxybutynin and (S)-oxybutynin, said TDDS releasing from 0.78 mg/24 h to 8 mg/24 h of said racemic (R/S)-oxybutynin or non-racemic mixture of (R)-oxybutynin and (S)-oxybutynin. The TDDS may also be formulated with a pharmaceutical carrier or vehicle.

The TDDS may contain a non-racemic mixture of (R)-oxybutynin and (S)-oxybutynin, in which (R)-oxybutynin is present from more than 50% to 95% by weight based on the total weight of the mixture. Preferably, the TDDS contains a mixture of (R)-oxybutynin and (S)-oxybutynin, in which (R)-oxybutynin is present from 50.01% to 95% by weight based on the total weight of the mixture.

The TDDS may contain a non-racemic mixture of (R)-oxybutynin and (S)-oxybutynin, in which (S)-oxybutynin is present from 5% to less than 50% by weight based on the total weight of the mixture. Preferably, the TDDS contains a mixture of (R)-oxybutynin and (S)-oxybutynin, in which (S)-oxybutynin is present from 5% to 49.99% by weight based on the total weight of the mixture.

The TDDS may contain a non-racemic mixture of (R)-oxybutynin and (S)-oxybutynin, in which (S)-oxybutynin is present from 40% or less by weight based on the total weight of the mixture.

The TDDS may contain a non-racemic mixture of (R)-oxybutynin and (S)-oxybutynin, in which (S)-oxybutynin is present from 30% or less by weight based on the total weight of the mixture.

The TDDS may contain a non-racemic mixture of (R)-oxybutynin and (S)-oxybutynin, in which (S)-oxybutynin is present from 20% or less by weight based on the total weight of the mixture.

The TDDS may contain a non-racemic mixture of (R)-oxybutynin and (S)-oxybutynin, in which (S)-oxybutynin is present from 10% or less by weight based on the total weight of the mixture.

According to another embodiment, the invention provides, a TDDS comprising (R)-oxybutynin, said TDDS releasing from 0.78 mg/24 h to 15.6 mg/24 h of said (R)-oxybutynin, preferably, from 0.78 mg/24 h to 4 mg/24 h of said (R)-oxybutynin. The TDDS may also be formulated with a pharmaceutical carrier or vehicle.

According to a further embodiment, the invention provides the use of a racemic (R/S)-oxybutynin or non-racemic mixture of (R)-oxybutynin and (S)-oxybutynin for the preparation of a medicament comprising said racemic (R/S)-oxybutynin or non-racemic mixture of (R)-oxybutynin and (S)-oxybutynin, in admixture with a pharmaceutical carrier, formulated in a TDDS, for the prevention and/or treatment of motion sickness.

According to a further embodiment, the invention provides the use of (R)-oxybutynin for the preparation of a medicament comprising said (R)-oxybutynin, in admixture with a pharmaceutical carrier, formulated in a TDDS, for the prevention or treatment of motion sickness.

According to yet a further embodiment, the use of a racemic (R/S)-oxybutynin or non-racemic mixture of (R)-oxybutynin and (S)-oxybutynin for the preparation of a medicament consisting of a pharmaceutical composition comprising, as an active ingredient, said racemic (R/S)-oxybutynin or non-racemic mixture of (R)-oxybutynin and (S)-oxybutynin, in admixture with a pharmaceutical carrier, formulated in a TDDS as described herein, for the prevention or treatment of motion sickness.

According to a yet further embodiment, the use of (R)-oxybutynin for the preparation of a medicament consisting of a pharmaceutical composition comprising, as an active ingredient, said (R)-oxybutynin, in admixture with a pharmaceutical carrier, formulated in a TDDS as described herein, for the prevention or treatment of motion sickness.

Said TDDS as described herein is preferably a patch, and utilizes a patch formulation wherein the aforementioned racemic (R/S)-oxybutynin or non-racemic mixture of (R)-oxybutynin and (S)-oxybutynin, is formulated with the conventional adjuvants such as D-sorbitol, gelatin, kaolin, methyl paraben, polysorbate 80, propylene glycol, propyl paraben, povidone, sodium carboxymethylcellulose, sodium polyacrylate, tartaric acid, titanium dioxide, and purified water. A patch formulation may also contain skin permeability enhancers such as lactate esters (e.g., lauryl lactate), triacetin or diethylene glycol monoethyl ether.

The TDDS-(R)-oxybutynin preferably is a patch, and utilizes a patch formulation wherein the aforementioned (R)-oxybutynin is formulated with the conventional adjuvants such as D-sorbitol, gelatin, kaolin, methyl paraben, polysorbate 80, propylene glycol, propyl paraben, povidone, sodium carboxymethylcellulose, sodium polyacrylate, tartaric acid, titanium dioxide, and purified water. A patch formulation may also contain skin permeability enhancer such as lactate esters (e.g., lauryl lactate), triacetin or diethylene glycol monoethyl ether.

The TDDS-racemic (R/S)-oxybutynin or TDDS-non-racemic (R)-oxybutynin and (S)-oxybutynin, for use according to a preferred embodiment of the present invention is a conventionally manufactured patch normally consisting of a matrix-type transdermal multilayer system comprising, at least, a polymeric film assuring the protection of the matrix; said matrix, consisting of at least one adhesive layer containing the above illustrated active ingredient in admixture with at least one of the above adjuvants, said layer being covered by a strip to be removed at the moment of the application of the patch, in order to allow the adhesive layer to be put in contact with the skin of the mammal for the prevention and/or treatment of motion sickness.

The TDDS-(R)-oxybutynin for use according to a preferred embodiment of the present invention is a conventionally manufactured patch normally consisting of a matrix-type transdermal multilayer system comprising, at least, a polymeric film assuring the protection of the matrix; said matrix, consisting of at least one adhesive layer containing the above illustrated active ingredient in admixture with at least one of the above adjuvants, said layer being covered by a strip to be removed at the moment of the application of the patch, in order to allow the adhesive layer to be put in contact with the skin of the mammal for the prevention and/or treatment of motion sickness.

In particular, a TDDS consisting of a patch is obtained as described for example in U.S. Pat. Nos. 5,212,199, 5,227,169, 5,747,065, 6,743,441, 7,081,249, 7,081,250, 7,081,251, 7,081,252, 7,087,241, US 2004/0057985 U.S. Pat. No. 8,420,117, US 2014/0271796, U.S. Pat. Nos. 8,802,134, 8,877,235, the disclosures of which are each incorporated herein by reference in their entirety. A TDDS consisting of non-occlusive topical formulation for transdermal administration of oxybutynin is obtained as described for example in EP 0966972, U.S. Pat. Nos. 4,889,845, 6,962,691, US 2003/0170194, US 2005/0064037, US 2006/0147383, U.S. Pat. Nos. 7,029,694, 7,179,483, US 2009/0018190, U.S. Pat. No. 8,241,662, US 2007/0225379, US 2010/216880, U.S. Pat. Nos. 8,652,491, 7,425,340, 7,214,381, 7,470,433, US 2008/0260842, US 2014/0037713, the disclosures of which are each incorporated herein by reference in their entirety. Typically, a TDDS in form of a solution, cream, lotion, spray, ointment, gel, is manufactured by mixing a predetermined amount of oxybutynin or of a pharmaceutically acceptable salt thereof; with common pharmaceutically acceptable carriers or vehicles and, optionally, with a permeation enhancer, of a gelling agent or thickening agent.

According to the present invention, the TDDS-racemic (R/S)-oxybutynin or TDDS-non-racemic (R)-oxybutynin and (S)-oxybutynin, to be used for said prevention and/or treatment may be in a range of released doses in order to accommodate differences in body weight and differences in severity of the syndrome in the subjects to be treated.

According to the present invention, the TDDS-(R)-oxybutynin, to be used for said prevention and/or treatment may be in a range of released doses in order to accommodate differences in body weight and differences in severity of the syndrome in the subjects to be treated.

Thus, the present invention provides a method for the prevention and/or treatment of motion sickness, which comprises applying to the skin of a subject in need of said prevention or treatment, a TDDS comprising a racemic (R/S)-oxybutynin or a non-racemic mixture of (R)-oxybutynin and (S)-oxybutynin.

In a racemic mixture of (R)-oxybutynin and (S)-oxybutynin, (R)-oxybutynin and (S)-oxybutynin are each present at 50% by weight based on the total weight of the mixture.

In a non-racemic mixture of (R)-oxybutynin and (S)-oxybutynin, (R)-oxybutynin is present from more than 50% to 95% by weight based on the total weight of the mixture and (S)-oxybutynin is present from 5% to less than 50% by weight based on the total weight of the mixture. Preferably, (R)-oxybutynin is present from 50.01% to 95% by weight based on the total weight of the mixture, and (S)-oxybutynin is present from 5% to 49.99% by weight based on the total weight of the mixture.

More particularly, the present invention provides a method for the prevention and/or treatment of motion sickness, which comprises applying to the skin of a human subject in need of said prevention and/or treatment a TDDS releasing from 0.78 mg/24 h to 15.6 mg/24 h, preferably from 0.78 mg/24 h to 8 mg/24 h, and preferably from 4 mg/24 h to 5 mg/24 h of (R)-oxybutynin in a non-racemic mixture of (R)-oxybutynin and (S)-oxybutynin.

In addition, the invention also provides a method for the prevention and/or treatment of motion sickness, which comprises applying to the skin of a human subject in need of said prevention and/or treatment a TDDS releasing from 0.78 mg/24 h to 31.2 mg/24 h, preferably from 0.78 mg/24 h to 15.6 mg/24 h, and preferably from 8 mg/24 h to 10 mg/24 h of a racemic (R/S)-oxybutynin.

The TDDS useful in the present method may contain a racemic mixture of (R)-oxybutynin and (S)-oxybutynin, in which (R)-oxybutynin and (S)-oxybutynin are each present at 50% by weight based on the total weight of the mixture.

The TDDS useful in the present method may contain a non-racemic mixture of (R)-oxybutynin and (S)-oxybutynin, in which (R)-oxybutynin is present from more than 50% to 95% by weight based on the total weight of the mixture. Preferably, the TDDS useful in the present method contains a mixture of (R)-oxybutynin and (S)-oxybutynin, in which (R)-oxybutynin is present from 50.01% to 95% by weight based on the total weight of the mixture.

The TDDS useful in the present method may contain a non-racemic mixture of (R)-oxybutynin and (S)-oxybutynin, in which (S)-oxybutynin is present from 5% to less than 50% by weight based on the total weight of the mixture. Preferably, the TDDS useful in the present method contains a mixture of (R)-oxybutynin and (S)-oxybutynin, in which (S)-oxybutynin is present from 5% to 49.99% by weight based on the total weight of the mixture.

The TDDS may contain (R)-oxybutynin present from 50% to 95% by weight based on the total weight of the mixture and (S)-oxybutynin present from 5% to 50% by weight based on the total weight of the mixture.

The TDDS may comprise a composition comprising (R)-oxybutynin present from 50% to 95% by weight based on the total weight of the mixture and (S)-oxybutynin present from 5% to 50% by weight based on the total weight of the mixture. The composition may further include a pharmaceutical carrier or vehicle.

The TDDS useful in the present method may contain a non-racemic mixture of (R)-oxybutynin and (S)-oxybutynin, in which (S)-oxybutynin is present from 40% or less by weight based on the total weight of the mixture.

The TDDS useful in the present method may contain a non-racemic mixture of (R)-oxybutynin and (S)-oxybutynin, in which (S)-oxybutynin is present from 30% or less by weight based on the total weight of the mixture.

The TDDS useful in the present method may contain a non-racemic mixture of (R)-oxybutynin and (S)-oxybutynin, in which (S)-oxybutynin is present from 20% or less by weight based on the total weight of the mixture.

The TDDS useful in the present method may contain a non-racemic mixture of (R)-oxybutynin and (S)-oxybutynin, in which (S)-oxybutynin is present from 10% or less by weight based on the total weight of the mixture.

A medicament useful in the present invention may consist of a pharmaceutical composition comprising (R)-oxybutynin present from 50% to 95% by weight based on the total weight of the mixture and (S)-oxybutynin present from 5% to 50% by weight based on the total weight of the mixture, formulated in a TDDS.

A pharmaceutical composition in dosage unit form comprising a mixture of (R)-oxybutynin, in a percent by weight of from 50.01% to 95% , and (S)-oxybutynin in a percent by weight of from 5% to 49.99% based on the total weight of said mixture, in admixture with a pharmaceutical carrier or vehicle.

A pharmaceutical composition in dosage unit form comprising a mixture of (S)-oxybutynin, in a percent by weight of from 50.01% to 95% , and (R)-oxybutynin in a percent by weight of from 5% to 49.99% based on the total weight of said mixture, in admixture with a pharmaceutical carrier or vehicle.

A pharmaceutical composition in dosage unit form comprising an active ingredient selected form the group consisting of
a mixture of (R)-oxybutynin, in a percent by weight of from 50.01% to 95%, and (S)-oxybutynin in a percent by weight of from 5% to 49.99% based on the total weight of said mixture; and
a mixture of (S)-oxybutynin, in a percent by weight of from 50.01% to 95%, and (R)-oxybutynin, in a percent by weight of from 5% to 49.99% the total weight of said mixture,

in admixture with a pharmaceutical carrier or vehicle.

In addition, the present invention provides a method for the prevention and/or treatment of motion sickness, which comprises applying to the skin of a mammalian subject in need of said prevention and/or treatment, a TDDS comprising (R)-oxybutynin.

Preferably, the present invention provides a method for the prevention and/or treatment of motion sickness, which comprises applying to the skin of a human subject in need of said prevention and/or treatment, a TDDS comprising (R)-oxybutynin.

The TDDS useful in the present method may contain (R)-oxybutynin and be free of (S)-oxybutynin.

The TDDS useful in the present method may contain (R)-oxybutynin and traces of (S)-oxybutynin.

More particularly, the present invention provides a method for the prevention and/or treatment of motion sickness, which comprises applying to the skin of a human subject in need of said prevention or treatment a TDDS releasing from 0.78 mg/24 h to 15.6 mg/24 h, advantageously from 0.78 mg/24 h to 4 mg/24 h, and preferably 4.8 mg-5.2 mg/24 h of the peripheral anticholinergic agent (R)-oxybutynin.

According to the present invention, the TDDS-oxybutynin to be used for said prevention and/or treatment may be in a range of released doses in order to accommodate differences in body weight and differences in severity of the syndrome in the subjects to be treated.

Thus, the present invention provides a method for the prevention and/or treatment of motion sickness, which comprises applying to the skin of a subject in need of said prevention or treatment a TDDS releasing from 0.78 mg/24 h to 31.2 mg/24 oxybutynin, advantageously from 0.78 mg/24 h to 8 mg/24 h, particularly preferably 3.9 mg/24 h, and particularly preferably 5.2 mg/24 h.

The present invention also provides a novel, TDDS comprising as an active ingredient, non-racemic mixture of (R)-oxybutynin and (S)-oxybutynin, said TDDS delivering (R)-oxybutynin in an amount from 0.78 mg/24 h to 15.6 mg/24 h, and normally from 0.78 mg/24 h to 4 mg/24 h of said active ingredient.

The present invention further provides a TDDS comprising as an active ingredient, racemic (R/S)-oxybutynin, said TDDS delivering said active ingredient in an amount from 0.78 mg/24 h to 31.2 mg/24 h, preferably from 0.78 mg/24 h to 15.6 mg/24 h, and more preferably from 0.78 mg/24 h to 8 mg/24 h of said active ingredient.

The present invention further provides a TDDS comprising an active ingredient, (R)-oxybutynin, said TDDS delivering said active ingredient in an amount from 0.78 mg/24 h to 15.6 mg/24 h, and preferably from 0.78 mg/24 h to 4 mg/24 h of said active ingredient.

According to an advantageous embodiment, the invention provides a TDDS comprising an active ingredient selected from the group consisting of (R)-oxybutynin, racemic (R/S)-oxybutynin, and non-racemic mixture of (R)-oxybutynin and (S)-oxybutynin, said TDDS delivering said active ingredient in an amount from 0.78 mg/24 h to 8 mg/24 h, and advantageously 5.2 mg (±0.5 mg)/24 h of said active ingredient.

### EXAMPLES

### Example 1

The efficacy of oxybutynin transdermal delivery system (TDDS) for the prevention of motion-induced sickness was tested in a simulated/analog environment in 12 healthy male volunteers. The primary objective of this randomized cross-over clinical trial was to demonstrate the reduction in the onset and severity of motion sickness following treatment with TDDS oxybutynin.

An optokinetics (OKN) drum was used to determine each subject's motion sickness tolerance. An OKN drum is a rotating drug delivery to test vision in which individuals are in a seated position facing the wall of the drum. The interior surface 15 of the drum is striped; thus, as the drum rotates, the subject's eyes are exposed to a moving visual field while the subject remains stationary. The OKN drum was set to rotate at 4 cycles per minute or 24 degrees per second. There are 20 black stripes in the 360 degree surround and 20 white stripes. In the participant field of vision, the nystagmus on the EOG will synchronize with the stripe rotation at about 1.3 Hz. The 8-foot tall drum from the seated participant perspective, completely fills the participant's field of view. A camera is mounted over the drum so the participant can be observed at all times for safety reasons. The test was administered approximately 24 hours after patch application.

The Karolinska Sleepiness Scale (KSS; Akerstedt and Gillberg M, 1990) was 25 used to assess subjective level of sleepiness. The KSS is a subject self-report measure of situational sleepiness and provides an assessment of alertness/sleepiness at a particular point in time. The KSS has been found to correlate with electroencephalogram and behavioral variables [Kaida et al., 2006). It is a 9-point categorical Likert scale where (1) is “extremely alert” and 9 is (9) “extremely 30 sleepy-fighting sleep.” Subjects were required to self-report their KSS assessments within 15 minutes prior to the OKN drum.

Twelve males (age>18 years) were enrolled in the study and allocated to TDDS oxybutynin (3.9 mg/24 hours) or to a placebo patch in a randomized crossover design. Each of the 2 treatment sessions was scheduled at least 1 week apart to prevent motion adaptation. On Days 1 and 8, subjects checked-in. Within 1 hour prior to dosing, subjects had vital signs taken while seated. Then, according to the randomization schedule they were assigned to, they were dosed with either one TDDS oxybutynin patch or 1 placebo patch. The patches were applied to the back on Days 1 and 8.

Approximately 23 hours post-patch application, subjects were required to arrive at the lab, where they had vital signs taken while in the seated position within 1 hour prior to entering the optokinetic (OKN) drum. They were also required to complete the Simulator Sickness Questionnaire (SSQ) within 30 minutes prior to the OKN drum and the Karolinska Sleepiness Scale (KSS) within 15 minutes prior to the OKN drum.

At approximately 24 hours post-patch application, subjects entered the OKN drum, where they remained for 32 minutes. A tape recording of the 16 items of the SSQ was played every 10 minutes while they were in the drum, for a total of 3 times during their 32 minutes in the drum. Subjects called out “None,” “Slight,” “Moderate,” or “Severe” in response to each item. A video recording was made for each person during their entire time in the drum to capture nystagmus. The oxybutynin or placebo patch were removed immediately following the OKN drum. At approximately 10, 30, and 60 minutes post-OKN drum, subjects were asked to complete the SSQ. Vital signs were measured at 60 and 120 minutes post-OKN drum while the subjects were in the seated position.

The primary efficacy outcome was the difference in peak SSQ score between transdermal oxybutynin and placebo. Results showed a clear reduction in the onset and severity of motion sickness following treatment with TDDS oxybutynin indicating that subjects' tolerance to motion sickness was better after treatment with TDDS oxybutynin compared to placebo.

No clinically relevant changes in blood pressure or heart rate were observed in the TDDS treatment group. Self-report of sleepiness showed no clinically relevant difference between TDDS oxybutynin and placebo.

In conclusion, TDDS oxybutynin is efficacious for the treatment of motion sickness with no significant cognitive or sedative effects.

The result of the above pilot study shows that, unexpectedly in respect of the current belief of the central origin of motion sickness, TDDS-oxybutynin is able to prevent and cure motion sickness. This is all the more surprising as it is well known that oxybutynin released from a TDDS-oxybutynin is not associated with Central Nervous side effects, suggesting a limited penetration of the blood brain barrier (BBB).

The above pilot study refers to an assay on adult voluntary human subjects, but the method of the present invention is applicable to the prevention and the cure of motion sickness in any human being, including infants and children, and also in animals, such as dogs and cats, that are particularly sensitive to this disorder.

### Example 2

That TDDS-(R)-oxybutynin prevents and treats motion sickness is demonstrated by a pilot study, per Example 1, using the commercially available oxybutynin patch releasing 3.9 mg/24 h of racemic oxybutynin, containing by definition a 50% amount of (R)-oxybutynin.

The efficacy of a higher dose of oxybutynin from a transdermal delivery system [TDDS]) releasing 7.8 mg of oxybutynin/24 hours for the prevention of motion-induced sickness was tested in a simulated/analog environment in up to 12 healthy male volunteers. The primary objective of this randomized cross-over clinical trial was to demonstrate the reduction in the onset and severity of motion sickness following treatment with TDDS oxybutynin.

An optokinetics (OKN) drum was used to determine each subject's motion sickness tolerance. An OKN drum is a rotating drug delivery to test vision in which individuals are in a seated position facing the wall of the drum. The interior surface of the drum is striped; thus, as the drum rotates, the subject's eyes are exposed to a moving visual field while the subject remains stationary. The OKN drum was set to rotate at 4 cycles per minute or 24 degrees per second. There are 20 black stripes in the 360 degree surround and 20 white stripes. In the participant field of vision, the nystagmus on the electrooculography (EOG) will synchronize with the stripe rotation at about 1.3 Hz. The 8 foot tall drum from the seated participant perspective, completely fills the participant's field of view. A camera is mounted over the drum so the participant can be observed at all times for safety reasons. The test was administered approximately 24 hours after patch application.

The Karolinska Sleepiness Scale (KSS; Akerstedt and Gillberg M, 1990) was used to assess subjective level of sleepiness. The KSS is a subject self-report measure of situational sleepiness and provides an assessment of alertness/sleepiness at a particular point in time. The KSS has been found to correlate with electroencephalogram and behavioral variables [Kaida et al, 2006). It is a 9-point categorical Likert scale where (1) is “extremely alert” and 9 is (9) “extremely sleepy-fighting sleep.” Subjects were required to self-report their KSS assessments within 15 minutes prior to the OKN drum.

Twelve males (age>18 years) were enrolled in the study and allocated to two TDDS oxybutynin (7.8 mg/24 hours) or to two placebo patch in a randomized cross-over design. Each of the 2 treatment sessions was scheduled at least 1 week apart to prevent motion adaptation. Subjects checked-in on the first day of each cross-over sequence. Within 1 hour prior to dosing, subjects had vital signs taken while seated. Then, according to the randomization schedule they were assigned to, they were dosed with either two TDDS oxybutynin patches or 2 placebo patches. The patches were applied to the back on Days 1 and 8.

Approximately 23 hours post-patch application, subjects were required to arrive at the lab, where they had vital signs taken while in the seated position within 1 hour prior to entering the optokinetic (OKN) drum. They were also required to complete the Simulator Sickness Questionnaire (SSQ) within 30 minutes prior to the OKN drum and the Karolinska Sleepiness Scale (KSS) within 15 minutes prior to the OKN drum.

At approximately 24 hours post-patch application, subjects entered the OKN drum, where they remained for 32 minutes. A tape recording of the 16 items of the SSQ was played every 10 minutes while they were in the drum, for a total of 3 times during their 32 minutes in the drum. Subjects called out “None,” “Slight,” “Moderate,” or “Severe” in response to each item. A video recording was made for each person during their entire time in the drum to capture nystagmus and as an additional safety measure. Immediately following the OKN drum, the oxybutynin or placebo patches were removed. At approximately 10, 30, and 60 minutes post-OKN drum, subjects were asked to complete the SSQ. Vital signs were measured at 60 and 120 minutes post-OKN drum while the subjects were in the seated position. The primary efficacy outcome was the difference in peak SSQ score between transdermal oxybutynin and placebo.

Results indicated that subjects' tolerance to motion sickness was better after treatment with TDDS oxybutynin compared to placebo. No clinically relevant changes in blood pressure or heart rate were observed in the TDDS treatment group. Self-report of sleepiness showed no clinically relevant difference between TDDS oxybutynin and placebo.

The above pilot study refers to an assay on adult voluntary human subjects, but the method of the present invention is applicable to the prevention and the cure of motion sickness in any human being, including infants and children, and also in animals, such as dogs, that are particularly sensitive to this disorder.

The foregoing detailed description has been given for illustration purposes only, especially for purposes of clarity of understanding. It will be apparent to those skilled in the art that certain changes and modifications may be practiced without departing from the spirit and scope of the invention, which is delineated by the appended claims.

### REFERENCES

Akerstedt T, Gillberg M. “Subjective and objective sleepiness in the active individual”. International Journal of Neuroscience, 1990; 52:29-37.

Bestaven E, Kambrun C, Guehl D, Cazalets J R, Guillaud E. “The influence of scopolamine on motor control and attentional processes”. nPeerJ 2016; May 5;4:e2008. doi: 10.7717/peerj.2008. eCollection 2016.

Brainard A., Gresham C. “Prevention and treatment of motion sickness”. Am Fam Physician 2014; July 1;90(1):41-6.

Estrada A, LeDuc PA, Curry I P, Phelps S E, Fuller D R. “Airsickness prevention in helicopter passengers”. Aviat Space Environ Med 2007; April;78(4):408-13.

Gil A, Nachum Z, Tal D, Shupak A. “A comparison of cinnarizine and transdermal scopolamine for the prevention of seasickness in naval crew: a double-blind, randomized, crossover study”. Clin Neuropharmacol 2012; January-February;35(1):37-9. doi: 10.1097/WNF.0b013e31823dc125.

Golding J F, Stott J R. “Comparison of the effects of a selective muscarinic receptor antagonist and hyoscine (scopolamine) on motion sickness, skin conductance and heart rate”. Br J Clin Pharmacol 1997; June;43(6):633-7.

Holland M S. “Central anticholinergic syndrome in a pediatric patient following transdermal scopolamine patch placement”. Nurse Anesth 1992; September;3(3):121-4.

Hulme et al 1978: Hulme E C, Birdsall N J, Burgen A S, Mehta P ( ) The binding of antagonists to brain muscarinic receptors. Mol Pharmacol 1978; 14: 737-750.

Kachur J F, Peterson J S, Carter J P, Rzeszotarski W J, Hanson R C, Noronha-Blob L. “R and S enantiomers of oxybutynin: pharmacological effects in guinea pig bladder and intestine”. J Pharmacol Exp Ther 1988; December;247(3):867-72.

Kaida et al. “Validation of the Karolinska sleepiness scale against performance and EEG variables.” Clinical Neurophysiology, (2006);117:1574-8].

Kennelly M J. “A comparative review of oxybutynin chloride formulations: pharmacokinetics and therapeutic efficacy in overactive bladder”. Rev Urol 2010;12(1):12-9.

Koslucher F, Haaland E, Malsch A, Webeler J, Stoffregen T A. “Sex Differences in the Incidence of Motion Sickness Induced by Linear Visual Oscillation”. Aerosp Med Hum Perform 2015; September;86(9):787-93. doi: 10.3357/AMHP.4243.2015.

Lin et al 2011: Lin Y -G, Chen P -H, Chang F -Y, 3 Wu L -T, Liao L -T, Tzee-Chung Wu T -Z. “Delirium due to Scopolamine Patch in a 4-Year-Old Boy”. Formos Med Assoc 2011;110(3):208-211.

Noronha-Blob L. Kachur J F. “Enantiomers of Oxybutynin : In vitro Pharmacological characterization at M₁, M₂ and M₃ Muscarinic Receptors and in Vivo Effects on Urinary Bladder Contraction, Mydriasis and Salvary Secretion in Guinea Pigs”. J Pharmacol Exp Ther 1991; 256:562-567. L. NORONHA-BLOB an

Parrot A C. “Transdermal scopolamine: a review of its effects upon motion sickness, psychological performance, and physiological functioning”. Aviat Space Environ Med 1989; January;60(1):1-9.

Patel and Ezzo 2009: Patel P N, Ezzo D. “Withdrawal symptoms after discontinuation of transdermal scopolamine therapy: Treatment with meclizine” Am J Health-System Pharmacy 2009, 66 (22) 2024-2026; DOI: https://doi.org/10.2146/ajhp080569.

Paule M G, Chelonis J J, Blake D J, Dornhoffer J L. “Effects of drug countermeasures for space motion sickness on working memory in humans”. Neurotoxicol Teratol 2004; November-December;26(6): 825-37.

Schmäl F. “Pharmacology. Neuronal mechanisms and the treatment of motion sickness” 2013;91(3-4):229-41. doi: 10.1159/000350185. Epub 2013; April 20.

Schmeller et al 1995: Schmeller T, Sporer F, Sauerwein M, Wink M ( ) Binding of tropane alkaloids to nicotinic and muscarinic acetylcholine receptors. Pharmazie 1995;50: 493-495.

Seo S W, Suh M K, Chin J, Na D L. “Mental confusion associated with scopolamine patch in elderly with mild cognitive impairment (MCI)”. Arch Gerontol Geriatr 2009; September-October;49(2):204-7.doi: 10.1016/j.archger.2008.07.011. Epub 2008; October 2.

Shupak A, Gordon C R, Spitzer O, Mendelowitz N, Melamed Y. “Three-years' experience of transdermal scopolamine: long-term effectiveness and side-effects”. Pharmatherapeutica 1989; 5(6):365-70.

Spinks A B, Wasiak J, Villanueva E V, Bernath V. “Scopolamine for preventing and treating motion sickness” Cochrane Database Syst Rev 2004; (3):CD002851.

Spinks A, Wasiak J. “Scopolamine (hyoscine) for preventing and treating motion sickness”. Cochrane Database Syst Rev 2011; June 15;(6):CD002851. doi: 10.1002/14651858.CD002851.pub4.

Zhang L L, Liu H Q, Yu X H, Zhang Y, Tian J S, Song X R, Han B, Liu A J. “The Combination of Scopolamine and Psychostimulants for the Prevention of Severe Motion Sickness”. CNS NeurosciTher 2016; August;22(8):715-22. doi: 10.1111/cns.12566. Epub 2016 May 9.

1. A method for preventing and/or treating motion sickness in a mammalian subject in need thereof, comprising administering to said mammalian, a transdermal drug delivery system (TDDS) comprising as an active ingredient, (R)-oxybutynin and (S)-oxybutynin.

2. A method for preventing or treating motion sickness in a mammalian subject in need thereof, comprising administering to said mammal, a transdermal drug delivery system (TDDS) comprising as an active ingredient, (R)-oxybutynin.

3. The method of claim 1 or 2, wherein said TDDS releases (R)-oxybutynin in an amount of from 0.78 mg/24 h to 15.6 mg/24 h.

4. The method of claim 1, wherein said TDDS releases (R)-oxybutynin in an amount of from 0.78 mg/24 h to 8 mg/24 h.

5. The method of claim 2, wherein said TDDS releases said active ingredient in an amount of from 0.78 mg/24 h to 4 mg/24 h.

6. The method of claim 1, wherein said (R)-oxybutynin and (S)-oxybutynin are present as a racemic mixture.

7. The method of claim 6, wherein said racemic mixture is released in an amount of from 0.78 mg/24 h to 31.2 mg/24 h.

8. The method of claim 2, wherein said TDDS is substantially free of (S)-oxybutynin.

9. The method of claim 8, wherein said TDDS is free of (S)-oxybutynin.

10. The method of claim 8, wherein said TDDS comprises traces of (S)-oxybutynin.

11. The method of claim 1 or 2, wherein the TDDS further comprises a pharmaceutical carrier or vehicle.

12. The method of claim 1 or 2, wherein the mammalian subject is a human.

13. A transdermal drug delivery system comprising (R)-oxybutynin, said transdermal drug delivery system releasing from 0.78 mg/24 h to 15.6 mg/24 h of said (R)-oxybutynin.

14. A transdermal drug delivery system comprising (R)-oxybutynin, said transdermal drug delivery system releasing from 0.78 mg/24 h to 4 mg/24 h of said (R)-oxybutynin.

15. A transdermal drug delivery system comprising a racemic mixture of (R)-oxybutynin and (S)-oxybutynin, said transdermal drug delivery system releasing from 0.78 mg/24 h to 31.2 mg/24 h of said racemic mixture of (R)-oxybutynin and (S)-oxybutynin.

16. A transdermal drug delivery system comprising a racemic mixture of (R)-oxybutynin and (S)-oxybutynin, said transdermal drug delivery system releasing from 0.78 mg/24 h to 8 mg/24 h of said racemic mixture of (R)-oxybutynin and (S)-oxybutynin.

17. A transdermal drug delivery system comprising a non-racemic mixture of (R)-oxybutynin and (S)-oxybutynin, said transdermal drug delivery system releasing from 0.78 mg/24 h to 15.6 mg/24 h of (R)-oxybutynin in said non-racemic mixture of (R)-oxybutynin and (S)-oxybutynin.

18. A transdermal drug delivery system comprising a non-racemic mixture of (R)-oxybutynin and (S)-oxybutynin, said transdermal drug delivery system releasing from 0.78 mg/24 h to 8 mg/24 h of (R)-oxybutynin in said non-racemic mixture of (R)-oxybutynin and (S)-oxybutynin.

19. The transdermal drug delivery system of claim 13, 14, 15, 16, 17, or 18, further comprising a pharmaceutical carrier or vehicle.

20. The transdermal drug delivery system of claim 13, 14, 15, 16, 17, or 18, wherein said transdermal drug delivery system is a patch, a patch pump, an infusion pump, or a micropump.

21. The transdermal drug delivery system of claim 13 or 14, wherein said TDDS is free of (S)-oxybutynin.

22. The transdermal drug delivery system of claim 13 or 14, wherein said TDDS comprises traces of (S)-oxybutynin.

23. The transdermal drug delivery system of claim 17 or 18, wherein (R)-oxybutynin is present from more than 50% to 95% by weight based on the total weight of the mixture.

24. The transdermal drug delivery system of claim 17 or 18, wherein (S)-oxybutynin is present from 5% to less than 50% by weight based on the total weight of the mixture.

25. The transdermal drug delivery system of claim 24, wherein (S)-oxybutynin is present from 40% or less by weight based on the total weight of the mixture.

26. The transdermal drug delivery system of claim 24, wherein (S)-oxybutynin is present from 30% or less by weight based on the total weight of the mixture.

27. The transdermal drug delivery system of claim 25, wherein (S)-oxybutynin is present from 20% or less by weight based on the total weight of the mixture.

28. The transdermal drug delivery system of claim 24, wherein (S)-oxybutynin is present from 10% or less by weight based on the total weight of the mixture.
